(19) United States
(12) Patent Application Publication
(10) Pub. No.: US 2022/0362927 A1
(43) Pub. Date: Nov. 17, 2022

(54) MOTION ASSIST DEVICE

(71) Applicant: Toyoflex Corporation, Tokyo (JP)

(72) Inventor: Ichiro CHIKANO, Tokyo (JP)

(73) Assignee: Toyoflex Corporation, Tokyo (JP)

(21) Appl. No.: 17/878,052

(22) Filed: Aug. 1, 2022

Related U.S. Application Data
(63) Continuation of application No. PCT/JP2021/003958, filed on Feb. 3, 2021.

(30) Foreign Application Priority Data
Feb. 6, 2020 (JP) 2020-019030

(51) Int. Cl. B25J 9/00; A61H 3/00

(52) U.S. Cl. CPC B25J 9/0006; A61H 3/00; A61H 2201/165; A61H 2201/1642

(57) ABSTRACT

A motion assisting device assists a movement of a wearer raising an upper body and includes a first assisting unit and a second assisting unit. The first assisting unit and the second assisting unit are attached to a left side surface and a right side surface of the wearer, respectively, and each include a power unit that swings a leg rod and applies, to the leg rod, a force in accordance with the swinging movement of the leg rod. The power unit is connected to a back surface frame that is a rigid body and connects the first assisting unit and the second assisting unit. A back plate of an upper body fixing member is connected to the back surface frame by a coupling member. The back plate is swingable at least in an up-down direction and a left-right direction with respect to the back surface frame.

### CROSS-REFERENCE TO RELATED APPLICATIONS

This application is a continuation application of International Application No. PCT/JP2021/003958, filed Feb. 3, 2021, which claims priority to Japanese Patent Application No. 2020-019030, filed Feb. 6, 2020. The contents of these applications are incorporated herein by reference in their entirety.

### BACKGROUND

Disclosed embodiments relate to a motion assisting device.

Japanese Patent Application Publication No. 2018-187709 discloses an arm and waist support device including an upper body frame to be mounted on a back of a wearer and a thigh arm to be mounted on a lower limb of the wearer. The arm and lower back support device applies a support force in a direction in which the upper body of the wearer is raised by urging the upper body frame toward the thigh arm.

### SUMMARY

However, in the arm and waist support device described in Japanese Patent Application Publication No. 2018-187709 involves a problem that the upper body frame does not easily follow a movement of the upper body of the wearer since the upper body frame is fixed to the back of the wearer.

Therefore, the present disclosure is directed to providing a motion assisting device capable of sufficiently following a movement of an upper body of a wearer.

A motion assisting device according to one embodiment of the present disclosure is configured to assist a movement of a wearer raising an upper body and includes: a first assisting unit and a second assisting unit, a leg rod, a waist fixing member, a back surface frame, an upper body fixing member, and a coupling member. The first assisting unit and a second assisting unit are respectively configured to be attached to a left side surface and a right side surface of the wearer when the motion assisting device is worn. The leg rod is provided in each of the first assisting unit and the second assisting unit and configured to be attached to a leg of the wearer when the motion assisting device is worn. The waist fixing member connects the first assisting unit and the second assisting unit. The waist fixing member is configured to be fixed to a waist of the wearer when the motion assisting device is worn. The back surface frame is a rigid body that connects the first assisting unit and the second assisting unit. The back surface frame protrudes upward from the first assisting unit and the second assisting unit and is configured to be arranged along a back of the wearer when the motion assisting device is worn. The upper body fixing member is configured to be fixed to the upper body of the wearer when the motion assisting device is worn. The upper body fixing member includes a back plate having a flat plate shape and is configured to abut against the back of the wearer when the motion assisting device is worn. The coupling member connects the back surface frame and the back plate. The first assisting unit and the second assisting unit each include a power unit that is configured to swing the leg rod and to apply a force to the leg rod in accordance with a swinging movement of the leg rod. The back surface frame is connected to the power unit. The coupling member is configured to be swingable at least in an up-down direction and a left-right direction with respect to the back surface frame.

### BRIEF DESCRIPTION OF DRAWINGS

FIG. 1 is a perspective view of a motion assisting device.

FIG. 2 is a front view of the motion assisting device.

FIG. 3 is a right side view of the motion assisting device.

FIG. 4 is a diagram illustrating a state where a back plate and a shoulder belt move (turn) in a left-right direction with respect to a back surface frame.

FIG. 5 is a diagram illustrating a state where the back plate and the shoulder belt move (turn) in an up-down direction with respect to the back surface frame.

FIG. 6 is a diagram illustrating a state where the back plate and the shoulder belt rotate with respect to the back surface frame.

FIG. 7 is a left side view illustrating an outline of an assisting unit and a leg rod.

FIG. 8 is a perspective view illustrating an outline of a power unit.

FIG. 9 is a perspective view illustrating an outline of the power unit.

FIGS. 10A to 10D are diagrams illustrating an outline of a large gear, in which FIG. 10A is a plan view seen from a +y direction, FIG. 10B is a perspective view, and FIGS. 10C and 10D are side views.

FIG. 11 is a diagram illustrating a state of the motion assisting device (assisting unit) when a wearer is walking with a leg positioned rearward.

FIG. 12 is a diagram illustrating a state of the motion assisting device (assisting unit) when the wearer slightly bends a waist.

FIG. 13 is a diagram illustrating a state of the motion assisting device (assisting unit) when the wearer further bends the waist.

FIGS. 14A and 14B are diagrams illustrating a state of a first pulley, a second pulley, and a cord-like body, in which FIG. 14A is a view seen from a +y side, and FIG. 14B is a view seen from a −x side.

FIGS. 15A and 15B are diagrams illustrating a state of the first pulley, the second pulley, and the cord-like body, in which FIG. 15A is a view seen from the +y side, and FIG. 15B is a view seen from the −x side.

FIGS. 16A to 16D are diagrams illustrating an outline of the first pulley, in which FIG. 16A is a plan view seen from the +y direction, FIG. 16B is a perspective view, and FIGS. 16C and 16D are side views.

FIGS. 17A to 17D are diagrams illustrating an outline of the second pulley, in which FIG. 17A is a plan view seen from the +y direction, FIG. 17B is a perspective view, and FIGS. 17C and 17D are side views.

### DETAILED DESCRIPTION OF EMBODIMENTS

Embodiments of the present disclosure will be described in detail below with reference to the drawings. A motion assisting device according to the present embodiment uses a cord-like body to assist a wearer in a movement of raising an upper body (a movement of stretching a waist).

A motion assisting device which is an example of the present embodiments will be described with reference to the drawings.

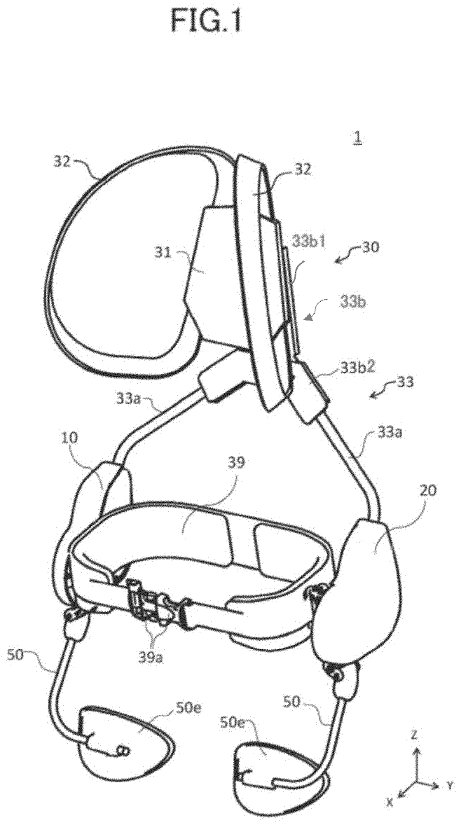

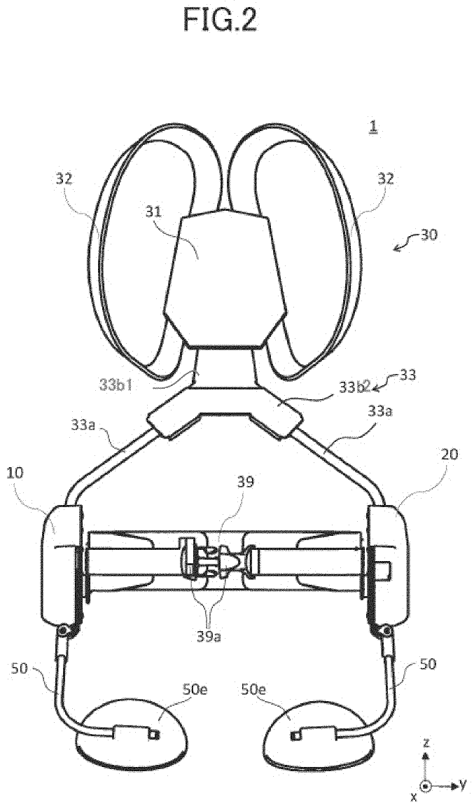

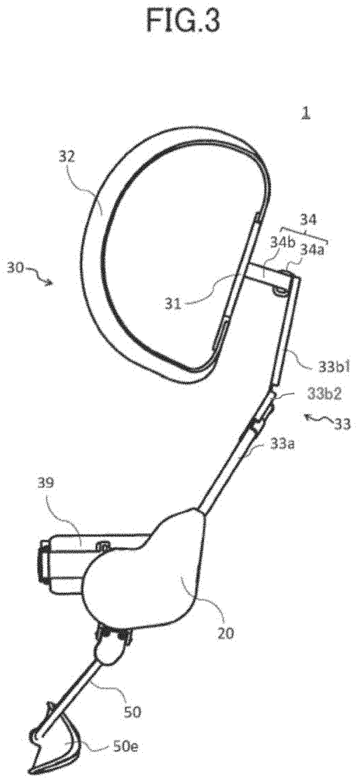

FIG. 1 is a perspective view of the motion assisting device, FIG. 2 is a front view thereof, and FIG. 3 is a right side view. A motion assisting device 1 is attached to cover a back of the wearer from both sides of the wearer. A front side of the wearer and a front side of the motion assisting device 1 are oriented in the same direction. Hereinafter, it is assumed that a direction from the rear to the front of the wearer is a +x direction, a direction from the right to the left of the wearer is a +y direction, and a direction from the bottom to the top of the wearer is a +z direction.

The motion assisting device 1 mainly includes an assisting unit 10, e.g., a right side assisting base, attached to a right side surface of the wearer, an assisting unit 20, e.g., a left side assisting base, attached to a left side surface of the wearer, an upper body fixing member 30, e.g., an upper body fixing mount, which is a member for attaching the assisting units 10 and 20 to the wearer, a back surface frame 33, a connection member 34, e.g., a connector, (see FIG. 3, corresponding to an example of a “coupling member”), a waist fixing member 39, e.g., a waist belt, and leg rods 50.

Each of the assisting units 10 and 20 includes a power unit 40, e.g., a power supply, (described in detail later) that swings the leg rod 50 and applies a force to the leg rod 50 in accordance with the swinging of the leg rod 50. The leg rods 50 are provided on the assisting units 10 and 20. The assisting units 10 and 20 will be described in detail later.

The upper body fixing member 30 is a member to be fixed to an upper body of the wearer when the motion assisting device 1 is worn. The upper body fixing member mainly includes a back plate 31 and a shoulder belt 32.

The back plate 31 is a plate-shaped member abutting against the back of the wearer when the motion assisting device 1 is worn. In the back plate 31, a surface abutting against the back of the wearer, that is, a surface on the front surface side of the motion assisting device 1 may be formed of a soft material having elastic force. Therefore, the back plate 31 does not bite into the back when the motion assisting device 1 is worn, and provides a good wearing feeling. The shape of the back plate 31 is not limited to the illustrated configuration.

The shoulder belt 32 is provided on the back plate 31 and is a member fixed to a shoulder portion of the wearer when the motion assisting device 1 is worn. In the present embodiment, the shoulder belt 32 includes a pair of band-shaped members formed of a soft material having elastic force and an adjustment mechanism, so that the shoulder belt 32 can be deformed following a movement of the wearer. The length of the shoulder belt 32 can be adjusted by the adjustment mechanism. The wearer shoulders the back plate 31 to position the back plate 31 along the back of the wearer, and then attaches the shoulder belt 32 to the shoulders to cause an inner surface of the shoulder belt 32 to abut against a body of the wearer. The shoulder belt 32 may be replaced by a chest belt fixed to a chest portion of the wearer when the motion assisting device 1 is worn.

The back surface frame 33 is provided on the assisting units 10 and 20, and protrudes upward from the assisting units 10 and 20. The back surface frame 33 is arranged along the back of the wearer when the motion assisting device 1 is worn.

The back surface frame 33 is symmetrical in the left-right direction and mainly includes rod-shaped portions 33a, e.g., a pair of rods to be connected to respective first and second assisting units 10, 20, and a plate-shaped portion 33b, e.g., a plate 33b1 and branches 33b2 extending therefrom to be connect to the rod-shaped portions 33a. The rod-shaped portions 33a may be rigid bodies formed by curving a rod material such as a hollow round rod. A first end of the rod-shaped portions 33a is connected to the assisting unit 10 or the assisting unit 20 (that is, the power unit 40 (described in detail later)), and a second end is arranged in the plate-shaped portion 33b, i.e., in the arms 33b2. The plate-shaped portion 33b may be a rigid body formed by bending a plate-shaped member. The rod-shaped portions 33a are provided at a lower end of the plate-shaped portion 33b, e.g., the branches 33b2, to be connected thereto. The plate 33b1 and branches 33b2 may be integral or may be separate and secured together.

Since the assisting unit 10 and the assisting unit 20 are connected by the back surface frame 33 that is a rigid body, the twisting of the assisting units 10 and 20 is suppressed even if a force that twists the assisting unit 10 and the assisting unit 20 against each other is generated. Therefore, an assisting force of the assisting units 10 and 20 can be surely transmitted to the wearer.

The form of the back surface frame 33 is not limited thereto. For example, a back surface frame may be formed by curving a rod material such as a hollow round rod into a substantially Y shape.

The connection member 34 is connected to an upper end of the back surface frame 33. The connection member 34 is connected to the upper body fixing member 30 and the back surface frame 33 while maintaining a constant distance between them. The connection member 34 swingably connects the upper body fixing member 30 and the back surface frame 33, so that the upper body fixing member 30 is caused to follow the movement of the upper body of the wearer. As may be seen, e.g., in FIG. 3, the connection member 34 may be connected to a center of the back plate 31 and an upper end of the plate 33b1, allowing more freedom of movement for a wearer. The plate 33b1 may extend further along the z direction towards a bottom of a wearer than the back plate 31. The connection member 34 will be described in detail later.

The waist fixing member 39 is a single band-shaped member, and connects the assisting unit 10 and the assisting unit 20. The waist fixing member 39 is fixed to a waist of the wearer when the motion assisting device 1 is worn, and thus, the assisting units 10 and 20 are held in the vicinity of the waist of the wearer. A pair of fixing portions 39a are fixed to a front surface side of the waist fixing member 39, and the waist fixing member 39 is held in a tubular shape by the fixing portions 39a. An inner peripheral length of the waist fixing member 39 is adjustable. The waist fixing member 39 may be a soft member that can deform following the movement of the wearer, so that the wearer can move easily.

The form of the waist fixing member 39 is not limited thereto. For example, a waist fixing member may be formed of a hard material, and an undeformable waist fixing member may be used to attach the assisting units 10 and 20 to side surfaces of the wearer.

Next, the connection member 34 will be described. As illustrated in FIG. 3, the connection member 34 mainly includes a metal fitting portion 34a, e.g., a pivot, and a string-shaped portion 34b, e.g., a beam.

The metal fitting portion 34a may be an annular member (ring) formed of a metal or a resin in a ring shape, and is provided swingably in the left-right direction on the back surface frame 33. The metal fitting portion 34a may be formed by bending a wire rod into a rectangular shape (here, a substantially rectangular shape). A longitudinal direction of the metal fitting portion 34a is oriented along an up-down direction. One long side of the metal fitting portion 34a is inserted into a hole formed in the back surface frame 33, so that the metal fitting portion 34a can swing in the left-right direction with respect to the back surface frame 33. The string-shaped portion 34b is provided on the other long side of the metal fitting portion 34a.

The string-shaped portion 34b is formed in a ring shape by connecting both ends of a string (here, a flat string), and has flexibility. The string-shaped portion 34b may include an adjustment mechanism that allows a length of the string-shaped portion 34b to be adjusted.

The string-shaped portion 34b is inserted into holes provided in the metal fitting portion 34a and the back plate 31, and connects the metal fitting portion 34a (that is, the back surface frame 33) and the back plate 31. The string-shaped portion 34b is movable in the up-down direction along the long side of the metal fitting portion 34a, and is swingable with respect to the metal fitting portion 34a.

The metal fitting portion 34a is formed by bending a wire rod into a rectangular shape, and thus is detachable. Therefore, it is possible to exchange the connection member 34 with a different connection member 34 by exchanging the metal fitting portion 34a. For example, when the string-shaped portion 34b does not include the adjustment mechanism, the length of the string-shaped portion 34b can be changed by exchanging the connection member 34. The metal fitting portion 34a may have an attaching and detaching mechanism to the back surface frame 33.

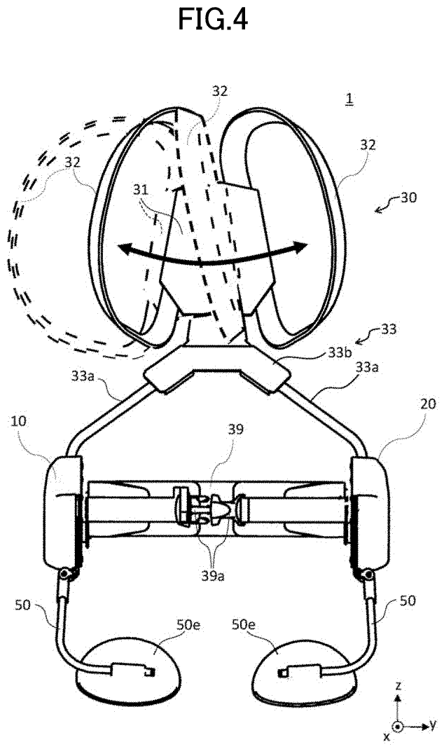

FIG. 4 is a diagram illustrating a state where the back plate 31 and the shoulder belt 32 move (turn) in the left-right direction with respect to the back surface frame 33. FIG. 4 illustrates a state before the back plate 31 and the shoulder belt 32 move by a solid line and a state where the back plate 31 and the shoulder belt 32 move in the −y direction by a dotted line. The metal fitting portion 34a (see FIG. 3) swings in the left-right direction with respect to the back surface frame 33, so that the back plate 31 and the shoulder belt 32 move in the left-right direction with respect to the back surface frame 33.

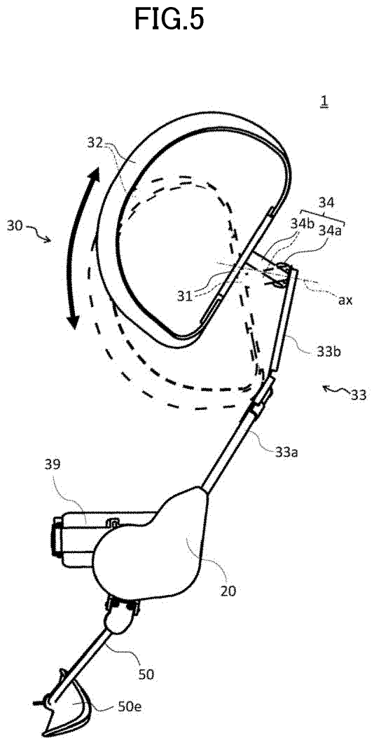

FIG. 5 is a diagram illustrating a state where the back plate 31 and the shoulder belt 32 move (turn) in the up-down direction with respect to the back surface frame 33. FIG. 5 illustrates a state where the back plate 31 and the shoulder belt 32 move upward by a solid line and a state where the back plate 31 and the shoulder belt 32 move downward by a dotted line. When the string-shaped portion 34b swings with respect to the metal fitting portion 34a, the back plate 31 and the shoulder belt 32 move in the up-down direction with respect to the back surface frame 33 about an axis ax. The axis ax is a virtual line passing through a center of the metal fitting portion 34a and being substantially orthogonal to a surface of the plate-shaped portion 33b at a position where the metal fitting portion 34a is provided.

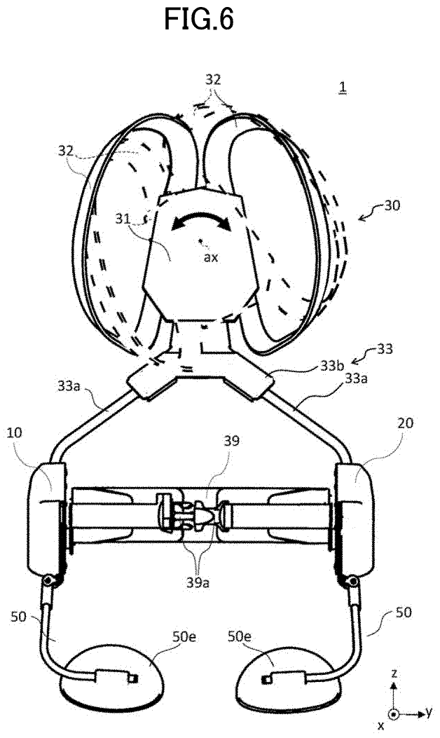

If a swinging movement in the left-right direction as illustrated in FIG. 4 and a swinging movement in the up-down direction as illustrated in FIG. 5 are combined, the back plate 31 and the shoulder belt 32 are movable in all directions with respect to the back surface frame 33. FIG. 6 is a diagram illustrating a state where the back plate 31 and the shoulder belt 32 rotate with respect to the back surface frame 33. Thus, the back plate 31 and the shoulder belt 32 can rotate about the axis ax.

The string-shaped portion 34b is flexible, and thus, the back plate 31 and the shoulder belt 32 can also rotate about the axis ax by a deformation of the string-shaped portion 34b.

Next, the assisting units 10 and 20 and the leg rod 50 will be described. The assisting unit 10 and the assisting unit 20 are symmetrical in the left-right direction, and thus, the configurations of the assisting units 10 and 20 will be described below with a focus on the assisting unit 20.

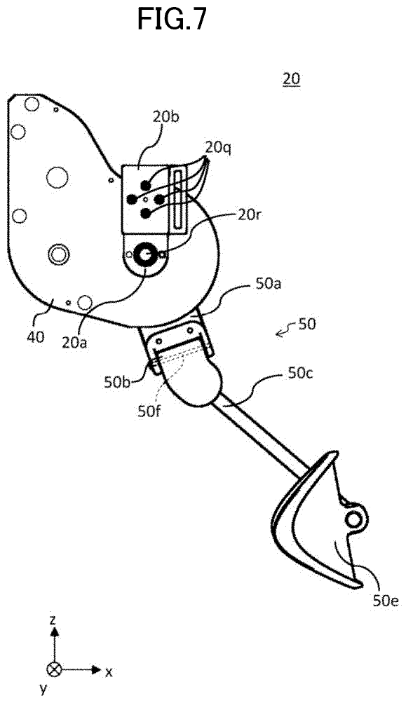

FIG. 7 is a left side view illustrating an outline of the assisting unit 20 and the leg rod 50. A connection portion 20a is connected to a left side surface of the assisting unit 20. The connection portion 20a is a flat plate-shaped member and is rotatable around a rotation shaft 20r.

The connection portion 20a is provided with a plate-shaped portion 20b having a flat plate shape. The waist fixing member 39 (see FIG. 6) is provided on the assisting unit 20 by sandwiching the waist fixing member 39 between the connection portion 20a and the plate-shaped portion 20b and fixing the connection portion 20a and the plate-shaped portion 20b with screws 20q.

The connection portion 20a and the plate-shaped portion 20b are interposed between the assisting unit 20 and the waist fixing member 39, and thus, the rotation shaft 20r and the waist fixing member 39 can be arranged at different positions. The assisting unit 20 and the waist fixing member 39 can rotate independently about the rotation shaft 20r without directly transmitting a rotational movement of one of the assisting unit 20 and the waist fixing member 39 to the other. The position of the rotation shaft 20r substantially coincides with a rotation position of a femoral portion of the wearer.

In the human body, the height in the z-axis direction differs between a rotation axis of the femoral portion and a rotation axis of the waist. Therefore, if the rotation shaft 20r of the assisting unit 20 and the waist fixing member 39 are arranged at substantially the same height, the waist fixing member 39 fails to follow a forward bending movement of the human body, which causes the waist fixing member 39 to twist. On the other hand, if the position of the waist fixing member 39 and the position of the rotation shaft 20r of the assisting unit 20 are different from each other so that the assisting unit 20 and the waist fixing member 39 is rotatable independently, the motion assisting device 1 can easily follow a forward bending movement of the human body.

The leg rod 50 is provided substantially along the femoral portion of the wearer, and mainly includes a rod connection portion 50a, a hinge 50b, a rod-shaped portion 50c, and a pad 50e. The leg rod 50 is attached to the femoral portion of the wearer via the pad 50e provided at a distal end of the leg rod 50. When the wearer causes the pad 50e to abut against a front side of the femoral portion of the wearer, a movement of the femoral portion is transmitted to the assisting units 10 and 20.

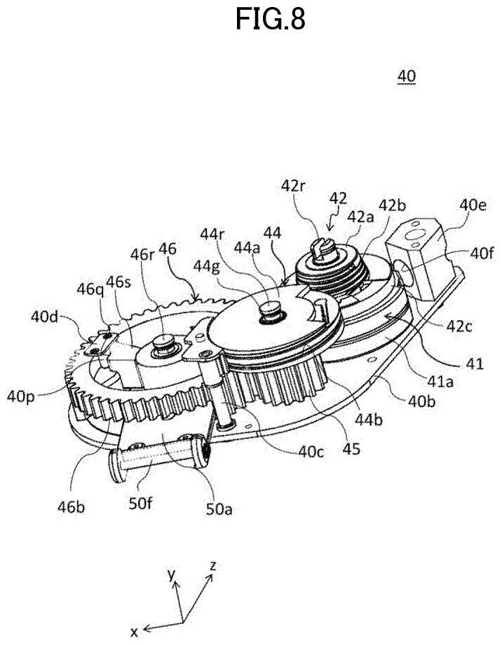

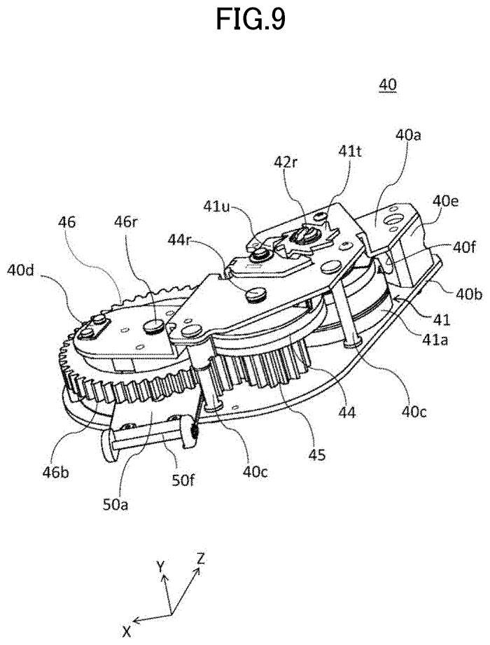

A first end of the rod connection portion 50a is connected to a rotation shaft 46r (see FIGS. 8, 9, and the like). The hinge 50b is provided at a second end of the rod connection portion 50a. The rod-shaped portion 50c is connected to the rod connection portion 50a via the hinge 50b. The hinge 50b includes a shaft 50f in a direction substantially orthogonal to the longitudinal direction of the rod connection portion 50a. Therefore, a movement of the pad 50e in an xz-plane is transmitted to the rod connection portion 50a, and a movement of the pad 50e in the y-axis direction is not transmitted to the rod connection portion 50a and is converted into a rotating movement of the hinge 50b.

FIGS. 8 and 9 are perspective views illustrating an outline of the power unit 40 included in the assisting unit 20. A first cover 40a is omitted from FIG. 8 for ease of viewing.

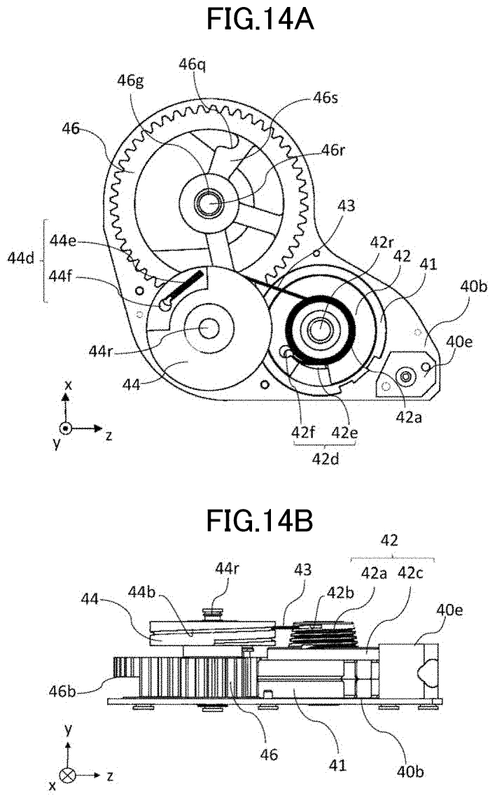

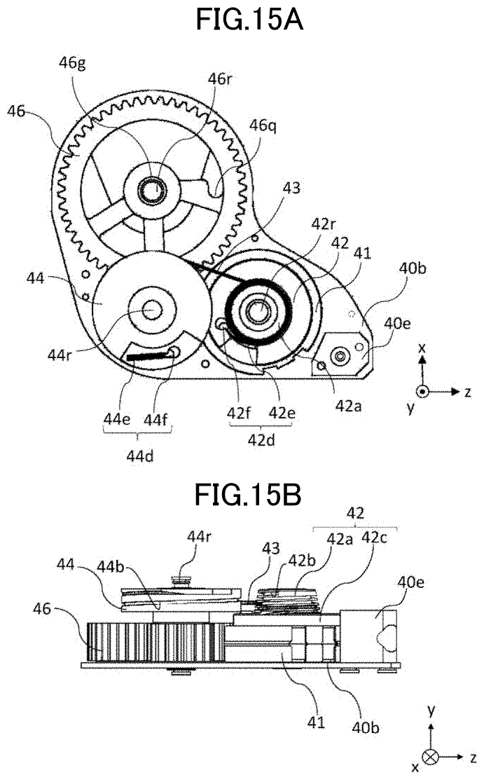

The power unit 40 mainly includes the first cover 40a, a second cover 40b, an elastic body unit 41, a first pulley 42, a cord-like body 43 (see FIGS. 14A and 14B, FIGS. 15A and 15B, and the like), a second pulley 44, a small gear 45, and a large gear 46.

The first cover 40a and the second cover 40b are substantially plate-shaped members forming outer edges on each of the ±y sides of the power unit 40, and are provided to face each other. Opposing ends of each of a plurality of columnar portions 40c having a cylindrical shape are connected to the first cover 40a and the second cover 40b, respectively. The columnar portions 40c maintain a gap between the first cover 40a and the second cover 40b. The elastic body unit 41, the first pulley 42, the cord-like body 43, the second pulley 44, the small gear 45, the large gear 46, and the rod connection portion 50a are provided between the first cover 40a and the second cover 40b.

The first cover 40a and the second cover 40b are provided with an attachment member 40e. The attachment member 40e is provided with a hole 40f into which the rod-shaped portion 33a is inserted. By inserting the rod-shaped portion 33a into the hole 40f and fixing the rod-shaped portion 33a and the attachment member 40e, the rod-shaped portion 33a, that is, the back surface frame 33 is connected to the power unit 40.

The elastic body unit 41, e.g., a force supply, may include a case 41a and an elastic body provided inside the case 41a. The elastic body may be a mainspring obtained by winding a plate-shaped member in a plane direction into a spiral shape. In the present embodiment, the mainspring is used as the elastic body, but the form of the elastic body is not limited thereto.

The first pulley 42 mainly includes a pulley main body 42a and a rotation shaft 42r. The first pulley 42 is rotatable about the rotation shaft 42r together with the rotation shaft 42r. An outer periphery of the pulley main body 42a has a substantially frustoconical shape gradually narrowing toward the distal end. A groove 42b having a spiral shape and having a width corresponding to a diameter of the cord-like body 43 is provided on the outer periphery of the pulley main body 42a.

The first pulley 42 is provided on the case 41a of the elastic body unit 41 via a base 42c. The rotation shaft 42r protrudes into the case 41a. A first end of the elastic body is provided in the rotation shaft 42r, and a second end is provided in the case 41a. Therefore, if the first pulley 42 rotates, the elastic body exerts, on the first pulley 42, an energizing force in accordance with a rotation angle of the first pulley 42.

As illustrated in FIG. 9, the rotation shaft 42r of the first pulley 42 is provided with a ratchet gear 41t on the outside (+Y side) of the first cover 40a. The first cover 40a is provided with a ratchet receiving portion 41u that meshes with a tooth of the ratchet gear 41t. A counterclockwise rotation of the ratchet gear 41t and the rotation shaft 42r is restricted by the ratchet receiving portion 41u. By the ratchet gear 41t and the rotation shaft 42r, the elastic body is wound by a constant number of rotations to be tightened even in an initial state (a state where the large gear 46 does not receive a load from the leg rod 50), and the wound and tightened state is maintained. Thus, the strength of an energizing force of the elastic body is adjustable.

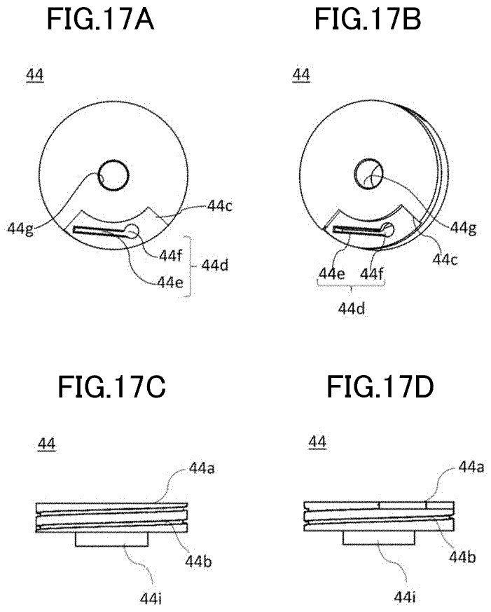

The second pulley 44 mainly includes a substantially disk-shaped pulley portion 44a in which the cord-like body 43 is wound around the outer periphery, and a thin cylindrical protruding portion 44i (see FIGS. 17A to 17D and the like) provided concentrically with the pulley portion 44a on a surface of the pulley portion 44a facing the −Y side. A groove 44b having a spiral shape and having a width corresponding to the diameter of the cord-like body 43 is provided on the outer periphery of the pulley portion 44a. A through-hole 44g through which a rotation shaft 44r is inserted is provided substantially in the center of the pulley portion 44a and the protruding portion 44i. The second pulley 44 is rotatable about the rotation shaft 44r.

A first end of the cord-like body 43 is provided in the first pulley 42 and a second end of the cord-like body 43 is provided in the second pulley 44, and the cord-like body 43 is housed in the groove 42b and the groove 44b. The arrangement and the like of the cord-like body 43 will be described in detail later.

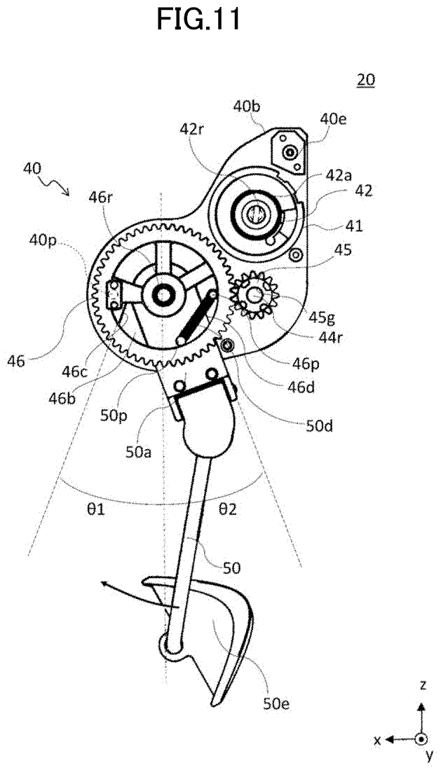

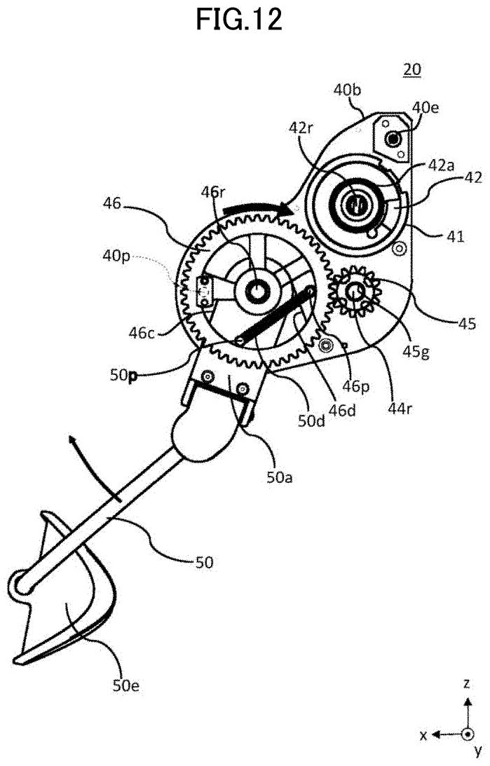

The small gear 45 is provided on the second pulley 44, and a through-hole 45g (see FIGS. 11, 12, and the like) through which the rotation shaft 44r is inserted is provided substantially in the center of the small gear 45. The small gear 45 rotates about the rotation shaft 44r together with the second pulley 44.

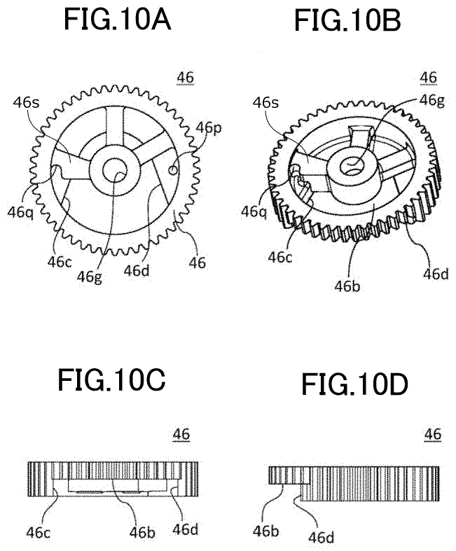

The large gear 46 is a gear having more teeth than the small gear 45. A through-hole 46g (see FIGS. 10A to 10D) through which the rotation shaft 46r is inserted is provided substantially in the center of the large gear 46, and the large gear 46 is rotatable about the rotation shaft 46r. The leg rod 50 is connected to the rotation shaft 46r and is rotatable about the rotation shaft 46r. The large gear 46 can be fitted with the small gear 45, so that the large gear 46 rotates with the rotation of the small gear 45.

A fixing plate 40d is screwed and fixed to the first cover 40a. The fixing plate 40d is provided with a large gear fixing pin 40p. As illustrated in FIG. 8, the large gear fixing pin 40p is provided to protrude in a direction (−y side) toward the second cover 40b, via a hole in the first cover 40a.

A spoke 46s of the large gear 46 is provided with a pin receiving portion 46q (see also FIGS. 10A and 10B). The pin receiving portion 46q is a recess portion having a substantially cylindrical shape, and extends along the y direction. The large gear fixing pin 40p can be fitted to the pin receiving portion 46q. Thus, it is possible to restrict a counterclockwise rotating movement of the large gear 46 when viewed from the +y direction.

FIGS. 10A to 10D are diagrams illustrating an outline of the large gear 46. FIG. 10A is a plan view seen from a +y direction. FIG. 10B is a perspective view. FIGS. 10C and 10D are side views. A surface of the large gear 46 on the −y side (a surface on the side to which the leg rod 50 is connected) is provided with a substantially fan-shaped recess 46b centered on the rotation shaft 46r and the through-hole 46g. The recess 46b includes side walls 46c and 46d.

Now, description returns to FIGS. 8 and 9. The rod connection portion 50a is an elongated flat plate-shaped member thinner than the depth of the recess 46b, and is rotatable inside the recess 46b along the plane of a bottom portion of the recess 46b. When the rod connection portion 50a and the side wall 46c abut against each other, the rotating movement of the leg rod 50 can be transmitted to the large gear 46, and the rotating movement of the large gear 46 can be transmitted to the leg rod 50.

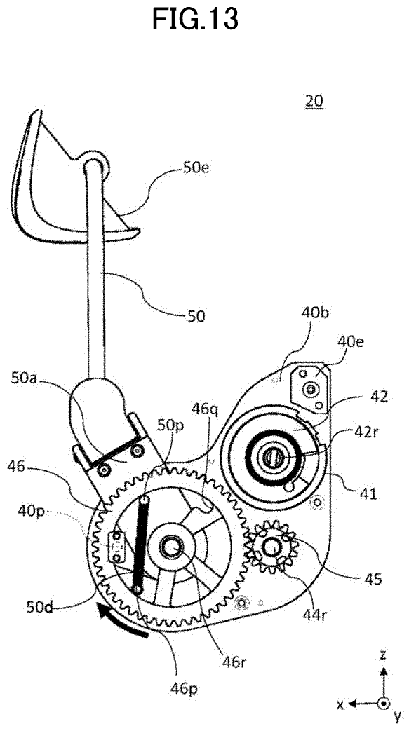

FIG. 11 is a diagram illustrating a state of the motion assisting device 1 (the assisting unit 20) when a wearer is walking with a leg positioned rearward. FIG. 12 is a diagram illustrating a state of the motion assisting device 1 (the assisting unit 20) when the wearer slightly bends the waist. FIG. 13 is a diagram illustrating a state of the motion assisting device 1 (the assisting unit 20) when the wearer further bends the waist. FIGS. 11 to 13 do not illustrate the cord-like body 43 and the second pulley 44 for ease of viewing.

The recess 46b is formed to be larger than the rod connection portion 50a so that the rod connection portion 50a can rotate inside the recess 46b. In the state illustrated in FIG. 9, the rod connection portion 50a is located furthermost to the −X side, and the rod connection portion 50a abuts against the side wall 46d.

As illustrated in FIGS. 11-13, the large gear 46 is provided with a pin 46p protruding in the +y direction. The rod connection portion 50a is provided with a pin 50p protruding in the +y direction. A lower rod energizing member 50d (for example, a tension coil spring) is provided between the pin 50p and the pin 46p, and an energizing force is applied to the rod connection portion 50a in a direction in which the rod connection portion 50a abuts against the side wall 46d (a direction in which the pad 50e is pressed against the leg of the wearer). In the state illustrated in FIG. 9, the large gear fixing pin 40p is fitted to the pin receiving portion 46q (not illustrated in FIG. 9), and thus, the large gear 46 does not rotate counterclockwise when viewed from the +y direction.

The rod connection portion 50a can idle inside the recess 46b between the side walls 46c and 46d. When the wearer starts bending the waist from the state illustrated in FIG. 11, the rod connection portion 50a idles clockwise (see the arrow in FIG. 11) inside the recess 46b. When the rod connection portion 50a rotates by a predetermined angle or more, the rod connection portion 50a abuts against the side wall 46c, as illustrated in FIG. 12.

From the state illustrated in FIG. 12, when the wearer further bends the waist and the rod connection portion 50a further rotates forward (clockwise in FIG. 12, see the arrow in FIG. 12), the rod connection portion 50a presses against the side wall 46c and applies a force in the rotation direction to the large gear 46 to rotate the large gear 46. As a result, the large gear 46 rotates clockwise (see the thick arrow in FIG. 12), the large gear fixing pin 40p is disengaged from the pin receiving portion 46q, and the state illustrated in FIG. 13 is obtained. That is, the power unit 40 switches between a first mode in which the rod connection portion 50a does not rotate the large gear 46 (the power unit 40 applies no force to the leg rod 50), and a second mode in which the rod connection portion 50a applies a force in the rotation direction to the large gear 46 to rotate the large gear 46 (the power unit 40 applies a force to the leg rod 50).

A switching angle between the first mode and the second mode and an idling angle of the leg rod 50 are determined by the side walls 46c and 46d. As illustrated in FIG. 11, the side wall 46c determines the switching angle between the first mode and the second mode on a front side of the wearer. The side walls 46c and 46d determine the idling angle of the leg rod 50 on the front side and the rear side of the wearer. In the present embodiment, an angle θ1 formed by a center line and the side wall 46c and an angle θ2 formed by the center line and the side wall 46d are substantially 20 degrees. However, the angles θ1 and θ2 are not limited to the illustrated aspects, as long as the angle θ1 is equal to or larger than the angle θ2. When the angle θ1 is equal to or larger than the angle θ2, the switching angle between the first mode and the second mode can be set to a size according to a walking movement.

The small gear 45 fitted to the large gear 46 rotates in accordance with the rotation of the large gear 46. Thus, the second pulley 44 rotates together with the small gear 45. The cord-like body 43 is pulled out from the first pulley 42 in accordance with the rotation of the second pulley 44, and rotates the first pulley 42.

Here, a configuration in which the first pulley 42 is rotated by the rotation of the second pulley 44 will be described. FIGS. 14A, 14B and FIGS. 15A, 15B are diagrams illustrating a state of the first pulley 42, the second pulley 44, and the cord-like body 43. FIGS. 14A and 15A are views seen from the +y side. FIGS. 14B and 15B are views seen from the −x side. FIGS. 14A and 14B illustrate a state where the cord-like body 43 is mainly wound around the first pulley 42. FIGS. 15A and 15B illustrate a state where the cord-like body 43 is wound around the first pulley 42 and the second pulley 44.

The cord-like body 43 is, for example, a wire rope, and the first end of the cord-like body 43 is connected to the first pulley 42 and the second end of the cord-like body 43 is connected to the second pulley 44. In the present embodiment, the wire rope is used as the cord-like body 43, but a string, a steel wire, and the like may be used as the cord-like body 43. A locking member (not illustrated) having a larger diameter than a middle part of the cord-like body 43 is connected to both ends of the cord-like body 43.

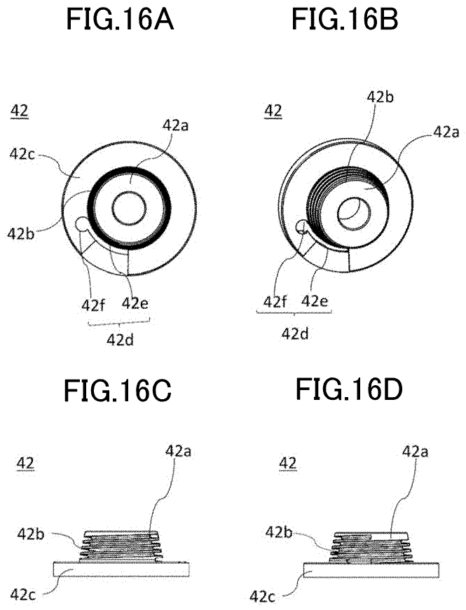

FIGS. 16A to 16D are diagrams illustrating an outline of the first pulley 42. FIG. 16A is a plan view seen from the +y direction. FIG. 16B is a perspective view. FIGS. 16C and 16D are side views. The first pulley 42 is provided with a cord-like body attachment portion 42d. The cord-like body attachment portion 42d mainly includes an elongated hole 42e and a circular hole 42f. When the locking member is fitted into the circular hole 42f and fixed by caulking or the like, one end of the cord-like body 43 is locked to the first pulley 42. An end portion of the cord-like body 43 is fixed to the cord-like body attachment portion 42d, is accommodated in the groove 42b in this state, and is spirally wound around the outer periphery of the pulley main body 42a.

FIGS. 17A to 17D are diagrams illustrating an outline of the second pulley 44. FIG. 17A is a plan view seen from the +y direction. FIG. 17B is a perspective view. FIGS. 17C and 17D are side views. The second pulley 44 is provided with a fan-shaped recess 44c. The recess 44c is provided with a cord-like body attachment portion 44d. The cord-like body attachment portion 44d mainly includes an elongated hole 44e and a circular hole 44f. When the locking member is fitted into the circular hole 44f and fixed by caulking or the like, one end of the cord-like body 43 is locked to the second pulley 44. An end portion of the cord-like body 43 is fixed to the cord-like body attachment portion 44d, is accommodated in the groove 44b in this state, and is spirally wound around the outer periphery of the pulley portion 44a.

Now, description returns to FIGS. 14A, 14B and FIGS. 15A, 15B. In FIGS. 14A and 14B, the second pulley 44 is not rotated, and thus, the cord-like body 43 is wound substantially around the entire first pulley 42, and is almost not wound around the second pulley 44 (the cord-like body 43 is substantially wound around about ¼ of the periphery of the second pulley 44). When the second pulley 44 rotates, the cord-like body 43 is unwound from the first pulley 42 and wound around the second pulley 44, as illustrated in FIGS. 15A and 15B.

The grooves 42b and 44b each have a spiral shape, and are arranged so that the cord-like body 43 is orthogonal to the rotation shaft 42r and the rotation shaft 44r, when the first pulley 42 and the second pulley 44 are viewed from the side. Therefore, it is possible to prevent the cord-like body 43 from being obliquely pulled out between the first pulley 42 and the second pulley 44, cause the cord-like body 43 to slide smoothly, and suppress loss in the transmission of force.

When the second pulley 44 rotates, the cord-like body 43 rotates the first pulley 42. However, the first pulley 42 is provided with the elastic body, and thus, the second pulley 44 and the first pulley 42 rotate against the energizing force of the elastic body. As the rotation amount of the first pulley 42 increases, the elastic force of the elastic body increases, and accordingly, the outer diameter of the pulley main body 42a of the first pulley 42 around which the cord-like body 43 is wound increases. Therefore, the force (apparent spring constant) required when the cord-like body 43 is unwound from the first pulley 42 (pulled out from the first pulley 42) can be kept substantially constant.

The outer diameter of the second pulley 44 is larger than the outer diameter of the first pulley 42, and thus, the energizing force of the elastic body is transmitted from the first pulley 42 to the second pulley 44, so that the torque is amplified between first pulley 42 and the second pulley 44. The outer diameter of the small gear 45 is smaller than the outer diameter of the second pulley 44, and the small gear 45 meshes with the large gear 46 having a large number of teeth, so that the torque is amplified between the second pulley 44 and the small gear 45, and the large gear 46. Therefore, an assisting force larger than the energizing force of the elastic body can be applied to the leg rod 50.

Now, description returns to FIGS. 11 to 13. When the wearer moves in a direction of bending the waist, the leg rod 50 and the large gear 46 rotate clockwise (see the arrows in FIGS. 11 to 13) against the energizing force of the elastic body, and by the rotation of the large gear 46, the small gear 45 and the second pulley 44 rotate against the energizing force of the elastic body. By the rotation of the second pulley 44, the cord-like body 43 is unwound from the first pulley 42 and wound around the second pulley 44, so that the first pulley 42 rotates against the energizing force of the elastic body.

When the wearer moves in a direction of stretching the waist, the elastic body exerts a restoring force that pulls the pulled out cord-like body 43 back to an original position. That is, when the first pulley 42 is rotated by the energizing force of the elastic body, the cord-like body 43 is wound around the pulley main body 42a, and the cord-like body 43 is unwound from the second pulley 44, the cord-like body 43 rotates the second pulley 44 and the small gear 45 and the large gear 46 each rotate by the rotation of the second pulley 44, so that the leg rod 50 returns to an original position.

According to the present embodiment, it is possible to assist a waist stretching movement when the wearer rises from a forward bending posture, by a simple configuration without using external motive power such as electricity. The energizing force of the elastic body can be amplified in two steps, and thus, it is possible to energize the leg rod 50 with a force larger than the energizing force of the elastic body. Therefore, an assisting force larger than the energizing force of the elastic body is generated, so that it is possible to efficiently assist a movement in which the waist of the wearer is returned to an upright state.

According to the present embodiment, since the back plate 31 and the back surface frame 33 are connected by the connection member 34, the upper body fixing member 30 (the back plate 31 and the shoulder belt 32) is movable with respect to the back surface frame 33. Therefore, the motion assisting device 1 can sufficiently follow the movement of the upper body of the wearer. Since the back plate 31 and the back surface frame 33 are separated, it is easy to attach the motion assisting device 1 to the wearer. In other words, only the back plate 31 that does not extend along an entirety of the back is in contact with the wearer, while the back surface frame 33 that connects to the first and second assisting units is not in contact with the wearer.

Since the back surface frame 33 is fixed to a dorsal portion for example, when the shoulder belt 32 is directly provided on the back surface frame 33, an upper body frame does not easily follow the movement of the upper body of the wearer. On the other hand, since the present embodiment involves that the back plate 31 and the connection member 34, and the plate-shaped portion 33b and the connection member 34 are each connected at one place, even if the wearer performs a movement such as twisting the upper body, the back plate 31 and the shoulder belt 32 move together with the upper body of the wearer, thereby providing good wearability. In the present embodiment, even if the wearer performs a movement such as twisting the upper body, the back surface frame 33 does not move together with the upper body. Therefore, the twisting of the assisting units 10 and 20 is suppressed, and the assisting force of the assisting units 10 and 20 can be surely transmitted to the wearer.

Since the string-shaped portion 34b is flexible, the string-shaped portion 34b is easily deformed when the wearer performs a movement such as twisting the upper body. Hence, discomfort can be reduced when the motion assisting device 1 is worn.

Since the back plate 31 and the back surface frame 33 are connected by the connection member 34, it is possible to apply the motion assisting device 1 to wearers having various body heights without changing the length of the connection member 34. When the length of the string-shaped portion 34b is adjustable, the heights of the back plate 31 and the shoulder belt 32 can be easily adjusted according to the body height of the wearer. Therefore, it is possible to easily apply the motion assisting device 1 to wearers having various body heights.

Changing the length of the string-shaped portion 34b makes it possible to change a bending angle of the waist of the wearer and a rotation angle of the leg rod 50. As a result, it is possible to adjust the assist force.

Since the leg rod 50 lays idle while the leg rod 50 is moving within a predetermined angle, for example, when the wearer is walking, assisting force does not occur. Therefore, the movement of the wearer is not hindered during the walking movement. Since the presence or absence of assistance is switched according to a relative angle between the leg rod 50 and the large gear 46, it is not necessary to perform a switching operation as to whether or not to generate an assisting force, so that the usability can be improved.

Although the present embodiment involves that the metal fitting portion 34a is a ring having a substantially rectangular shape, and the string-shaped portion 34b is a flat string, the form of portions 34a and 34b is not limited thereto. For example, the metal fitting portion 34a may be a ring having a substantially annular shape or a substantially elliptical ring shape, and the string-shaped portion 34b may be a round string. A cord-like body such as a rope or a cable can be used as the string-shaped portion 34b. Alternatively, the metal fitting portion may be replaced by a hole provided in the back surface frame 33 (here, the plate-shaped portion 33b), and the string-shaped portion 34b may be provided directly in the hole. For example, the simplest structure is a structure in which a rope is used as a string-shaped portion and the back plate 31 and the plate-shaped portion 33b are provided on both ends of the rope, so that the rope forms a connection member.

Although the present embodiment involves that the string-shaped portion 34b is flexible, the string-shaped portion may be a rigid body. For example, a part or the whole of the string-shaped portion may be formed of an elastic body such as rubber or a spring, so that the string-shaped portion is elastic. Even if the string-shaped portion is elastic, it is only required that the string-shaped portion 34b can move in the up-down direction along the long side of the metal fitting portion 34a, or that the string-shaped portion 34b is swingable with respect to the metal fitting portion 34a. If the string-shaped portion is elastic, even when an unexpected impact is applied to the assisting units 10 and 20 and the back surface frame 33, it is possible to absorb the impact and prevent a burden on the wearer.

Although the present embodiment performs switching between a first mode in which the rod connection portion 50a idles inside the recess 46b, so that the power unit 40 applies no force to the leg rod 50, and a second mode in which the power unit 40 applies force to the leg rod 50, the configuration for switching between the first mode and the second mode is not limited thereto. For example, the leg rod 50 and the large gear 46 may be connected by a cord-like body, and the first mode may be a range in which the cord-like body is not extended, and the second mode may be a state where the cord-like body is extended. For example, the leg rod 50 may be provided with a pin, the large gear 46 may be provided with an elongated hole through which the pin can slide, and the first mode may be a range in which the pin moves in the elongated hole, and the second mode may be a state where the pin abuts against an end of the elongated hole.

Thus, the present embodiments are described in detail with reference to the drawings. However, the specific configuration is not limited to the embodiment, and a change of the design and the like within the spirit of the disclosed embodiments is also included in the embodiment. The technical ideas of the disclosed embodiments may be applied to devices other than the motion assisting device that assists the movement of a human body.

In the present embodiments, the term “substantially” is a concept including not only exact uniformity, but also an error or a deformation to an extent at which sameness is not lost. For example, the terms “substantially parallel” and “substantially orthogonal” are not limited to cases where components are arranged exactly parallel or orthogonal. For example, even when simply the terms “parallel”, “orthogonal”, and the like are used, the concept includes not only exactly parallel or orthogonal arrangements, but also substantially parallel and substantially orthogonal arrangements. In the present embodiments, the term “vicinity” is a concept indicating that the vicinity of A includes a range near A that may or may not include A, for example.

What is claimed is:

1. A motion assisting device configured to assist a movement of a wearer raising an upper body, the motion assisting device comprising:
a first assisting base and a second assisting base respectively configured to be attached to a left side surface and a right side surface of the wearer when the motion assisting device is worn;
a leg rod provided in each of the first assisting base and the second assisting base and configured to be attached to a leg of the wearer when the motion assisting device is worn;
a waist belt that connects the first assisting base and the second assisting base, the waist belt being configured to be fixed to a waist of the wearer when the motion assisting device is worn;
a back surface frame that connects the first assisting base and the second assisting base, the back surface frame protruding upward from the first assisting base and the second assisting base and being configured to be arranged along a back of the wearer when the motion assisting device is worn;
an upper body fixing mount configured to be fixed to the upper body of the wearer when the motion assisting device is worn, the upper body fixing mount including a back plate configured to abut against the back of the wearer when the motion assisting device is worn; and
a connector that connects the back surface frame and the back plate, wherein
the first assisting base and the second assisting base each include a power supply that is configured to swing the leg rod and to apply a force to the leg rod in accordance with a swinging movement of the leg rod,
the back surface frame is connected to the power supply of each of the first assisting base and the second assisting base, and
the connector is configured to be swingable at least in an up-down direction and a left-right direction with respect to the back surface frame.

2. The motion assisting device according to claim 1, wherein the connector is elastic.

3. The motion assisting device according to claim 1, wherein the connector is flexible.

4. The motion assisting device according to claim 1, wherein a length of the connector is adjustable.

5. The motion assisting device according to claim 1, wherein the connector includes:
a pivot connected to the back surface frame, the pivot being swingable in a left-right direction in the back surface frame; and
a beam connected to the pivot and the back plate, the beam being swingable in an up-down direction that crosses the left-right direction.

6. The motion assisting device according to claim 1, wherein the power supply is configured to switch, in accordance with a swing angle of the leg rod, between a first mode in which the power supply applies a force to the leg rod and a second mode in which the power supply applies no force to the leg rod.

7. The motion assisting device according to claim 1, wherein the back surface frame includes a rigid body.

8. The motion assisting device according to claim 7, wherein the connector is connected to an upper end of the rigid body and a center of the back plate.

9. The motion assisting device according to claim 1, wherein the back surface frame includes a two rigid bodies, a first rigid body connected to the back plate and a second rigid body connected to the first rigid body and the first and second assisting bases.

10. The motion assisting device according to claim 9, wherein the first rigid body is a plate and the second rigid body includes two rods.

11. The motion assisting device according to claim 1, wherein the connector is connected to an upper end of the back surface frame and a center of the back plate.
